(19) United States
(12) Patent Application Publication
(10) Pub. No.: US 2011/0053276 A1
(43) Pub. Date: Mar. 3, 2011

(54) MOLECULAR INDICATOR AND PROCESS OF SYNTHESIZING

(76) Inventors: Donald W. Zehnder, II, Westerville, OH (US); John R. Shaw, Lewis Center, OH (US)

(21) Appl. No.: 12/674,239

(22) PCT Filed: Aug. 25, 2008
(86) PCT No.: PCT/US08/74192

§ 371 Date: Feb. 19, 2010

Related U.S. Application Data
(60) Provisional application No. 60/965,972, filed on Aug. 23, 2007.

(51) Int. Cl. G01N 33/02; C07D 323/00

(52) U.S. Cl. 436/20; 549/352; 534/798

(57) ABSTRACT

A molecular indicator for detecting primary diamines includes an indicator structure coupled to (a) a bis-crown ether structure or (b) a structure having a bis-crown ether-like functionality. A package for detecting an analyte emitted by a packaged object includes a packaging material for the object. The package further includes a molecular indicator included with the packaging material for detecting the analyte. The molecular indicator includes an indicator structure coupled to a trapping structure for trapping the analyte. The molecular indicator is highly specific for the analyte, and the trapping of the analyte by the molecular indicator avoids reactive chemistry by-products. The molecular indicator can be synthesized by several different process schemes.

### CROSS-REFERENCE TO RELATED APPLICATION

This application claims the benefit of U.S. Provisional. Application No. 60/965,972, filed Aug. 23, 2007, the disclosure of which is incorporated herein by reference.

### BACKGROUND OF THE INVENTION

The determination of whether food has spoiled is of interest to a wide-ranging group of people, from producers to consumers. This range includes producers, regulators, importers, exporters, traders, and transporters. Many food products can spoil; however, one of the greatest concerns is meat and meat products. Spoiled foods pose many risks, including potentially life threatening illnesses to the very young or elderly, as well as those with compromised immune systems. Food spoilage can be accelerated as a result of unusual circumstances in the packaging or distribution cycle. There are concerns over the use of modified atmosphere packaging and its impact on the apparent freshness of packaged meats. Food spoilage is typically monitored by standard analytical laboratory procedures involving microbiological and chemical analysis. These methods are not available to consumers at the point of purchase, nor do they generate real-time responses.

Colorimetric indicators have been developed for off-gas detection in applications such as individual protection (end-of-service life indicators for carbon filters), environmental detection, and Chemical sensors. Metalloporphyrins, acid base indicators, push pull chromophores, or host-guest complexes have shown utility for detecting specific chemical structures.

Various types of indicators have been proposed for use in food quality testing, including organometallic complexes, reactive chemistry, and pH indicators. These indicator chemistries are not appropriate for use as in-package indicators because of food safety concerns (they contain heavy metals or produce reactive chemistry by-products) or non-specific modes of detection (pH indicators) prone to interferences.

### SUMMARY OF THE INVENTION

In one embodiment, a molecular indicator for detecting primary diamines is provided. The molecular indicator includes an indicator structure coupled to (a) a bis-crown ether structure or (b) a structure having a bis-crown ether-like functionality.

In another embodiment, a package for detecting an analyte emitted by a packaged object is provided. The package includes a packaging material for the object. The package further includes a molecular indicator included with the packaging material for detecting the analyte. The molecular indicator includes an indicator structure coupled to a trapping structure for trapping the analyte. The molecular indicator is highly specific for the analyte, and the trapping of the analyte by the molecular indicator avoids reactive chemistry by-products.

In some further embodiments, processes of synthesizing a molecular indicator are provided.

### DETAILED DESCRIPTION OF THE PREFERRED EMBODIMENTS

A molecular indicator and a package including the molecular indicator are provided. The molecular indicator enables the package to detect an analyte emitted by an object contained within the package. The molecular indicator works on a “trap and detect” principle: the molecular indicator includes a trapping-structure for trapping the analyte, and an indicator structure coupled to the trapping structure which allows for detection of the molecular indicator with the trapped analyte.

By “trap” is meant that the trapping structure traps or sequesters the analyte by a method other than covalent bonding. The trapping structure of the molecular indicator is highly specific for the analyte. This means that the trapping structure has a specificity for the chemistry of the analyte to allow trapping or sequestering of the analyte, for example a specificity for a functional group of the analyte, and also that the trapping structure has a specificity for the size of the analyte to allow trapping or sequestering of the analyte.

The trapping of the analyte by the molecular indicator also avoids reactive chemistry by-products. There are no reactive chemistry byproducts with this trapping mechanism because it is not a reactive type bonding. Thus, the molecular indicator avoids specificity problems observed in other indicators (pH) and avoids reactive chemistry by-products, such that it can be used as an in-package indicator.

The molecular indicator may be used for trapping and detecting many different analytes associated with packaged objects. In a particular embodiment, the molecular indicator is used for detecting the primary by-products of meat spoilage, which are biogenic diamines such as cadaverine and putrescine. Alternatively, the molecular indicator could be used for detecting spoilage byproducts from other foods such as dairy or dry foods. The biogenic diamines from meat spoilage are primary diamines, but alternatively the molecular indicator could be used to trap and detect other functional groups such as sulfides, phosphonates, and secondary and tertiary amines.

Since the trapping structure of the molecular indicator is specific for the analyte, the molecular indicator can include a wide variety of different trapping structures depending on the particular analyte. In a particular embodiment, when the analyte is a primary diamine, the trapping structure is either (a) a bis-crown ether structure or (b) a structure having a bis-crown ether-like functionality. Structure 1 below shows an example of a molecular indicator which includes a bis-crown ether structure. A primary diamine is shown trapped by the bis-crown ether structure.

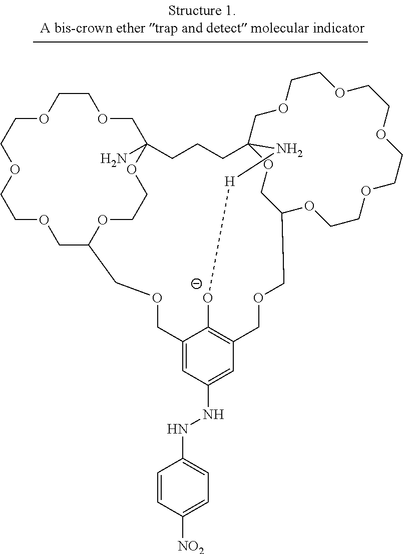

In the example shown in Structure 1, the primary diamine is entrapped via molecular interactions between the amines and two crown ethers. These molecular components comprise a “molecular trap”. The primary diamine is entrapped within the space allowed by the molecular trap and the coupled indicator structure (described below).

Alternatively, the molecular indicator can include a structure having a bis-crown ether-like functionality. For example, the oxygen atoms of a bis-crown ether could be replaced with other electronegative atoms such as sulfur or nitrogen atoms, while achieving a similar functionality in terms of trapping the analyte by electronic interactions. As another example, the ether ring can be modified to have different substituents on it. Also, other cyclic ethers besides crown ethers can be used. The process schemes at the end of the description show some different examples of structures having bis-crown ether-like functionality.

The molecular indicator includes an indicator structure coupled to the trapping structure. The indicator structure allows for detection of the molecular indicator with the trapped analyte. Any type of structure suitable for allowing detection can be used. In a particular example, the indicator structure interacts with the analyte to allow detection of the analyte. For example, when the molecular indicator is used to detect primary diamines, in some embodiments the indicator structure interacts via hydrogen bonding with the diamine. In some embodiments such indicators contain phenolic alcohols as molecular switches and photochromic moieties that effect color change upon detection of the analyte. Some examples of photochromic moieties include such functional groups as aromatic azo compounds, sulfonated hydroxyl-functional triphenylmethane dyes, and nitrothiophenes. In the example shown in Structure 1, the molecular indicator includes a phenolic azo-dye indicator coupled to the bis-crown ether, the indicator interacting with the diamine via hydrogen bonding.

The package further includes a packaging material for the object. The molecular indicator is included with the packaging material. More generally, the molecular indicator can be included with any suitable substrate such as packaging materials and other substrates. The molecular indicator can be included inside the substrate or on a surface of the substrate, and it can be held to the substrate by any suitable interaction. For example, in one embodiment, the molecular indicator is compounded with a polymer and is included inside the resulting polymer matrix. In a particular embodiment, the polymer matrix is a polymeric food package, and the resulting package detects meat spoilage via color change. Alternatively, the molecular indicator could be coated or otherwise held on the surface of the polymer. Some examples of different substrates can include paper, paperboard, polymer, plastic, cotton, resin, glass, fiberglass, or textile fabric.

One embodiment involves the incorporation of the molecular indicator in a porous or semipermeable substrate for the purposes of providing an in-package indicator, or an indicating package (e.g., food wrap or product tray). Incorporation of the indicator can be achieved either through chemical bonding and attachment to polymeric monomers and subsequent co-polymerization or dissolution of the indicator into polymeric solution and casting thin films. Examples of porous substrates include cellulose, non-woven fabrics, or polymer fibers. Examples of semipermeable materials include ethylene vinyl acetate, polyolefins (including polyethylene and polypropylene), polystyrene, polycarbonate, polytetrafluorethylene, fluorpolymers, polymethylmethacrylate, acetal, polyvinyl chloride, phenoxy, polyester, nylon, polyvinylidenefluoride, epoxy, polyvinylidinechloride, and nitriles. These materials may be used as single layer films or may be used together as multilayered films.

The packaging material or other substrate can include one or more additives to enhance the detection of the analyte. For example, an additive can be included in the substrate which improves the transport of the analyte through the substrate. The improved analyte transport through the substrate increases the concentration of analyte available for detection and thereby enhances the detection. Additives can be included that provide optimized transport, solubility or colorimetric properties. Some examples of additives that may improve one or more of these properties include cucurbiturils, cyclodextrins, silicon dioxide, and mineral clays.

The molecular indicator may be useful in many different applications and industries. For example, while a particular embodiment relates to the use of the molecular indicator in the meat packaging industry for detecting spoilage gases from meat products, the molecular indicator may also be useful in other food packaging businesses. The molecular indicator may be incorporated in many different types of food packaging materials, such as paper, paperboard, polymer films, or styrofoam. The molecular indicator may be used in packaging in the retail, distribution and storage containers.

The molecular indicator can be synthesized by any suitable processes. A first process for synthesizing the molecular indicator is shown by the following steps in Scheme 1.

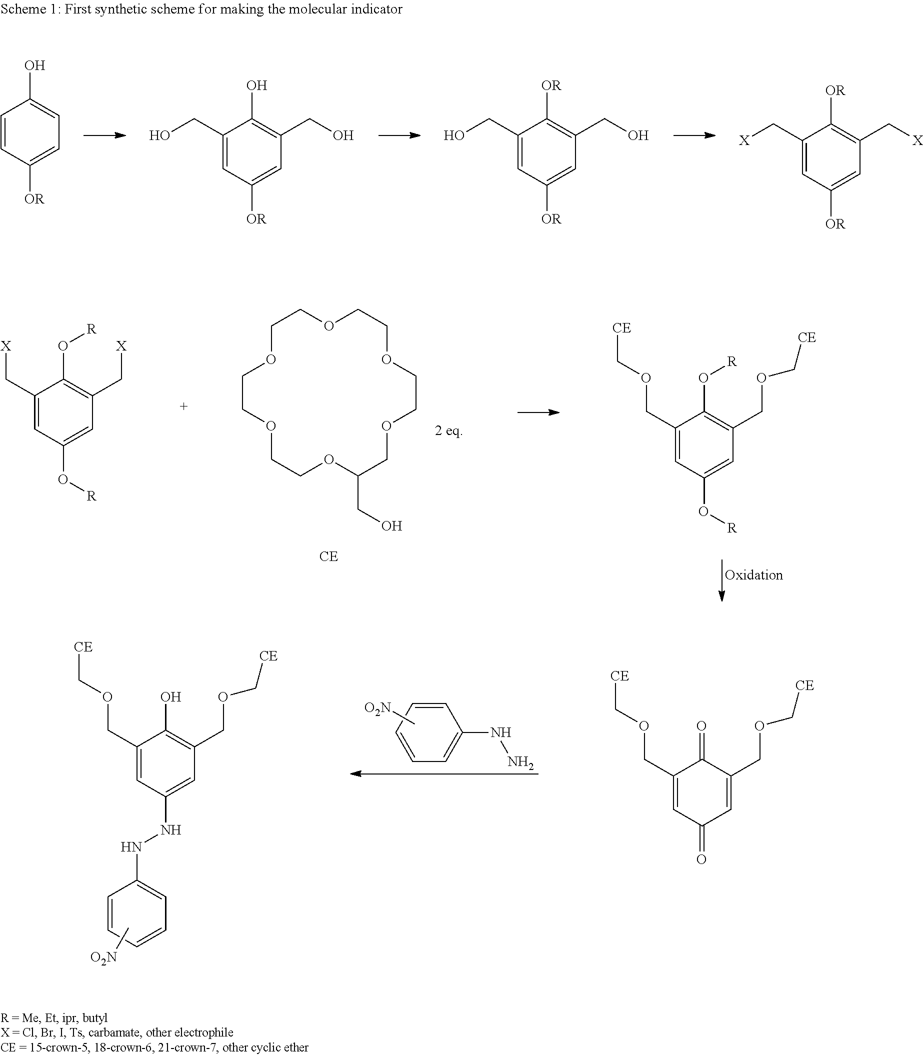

The following Scheme 1A shows an example of the first synthetic scheme for making the molecular indicator.

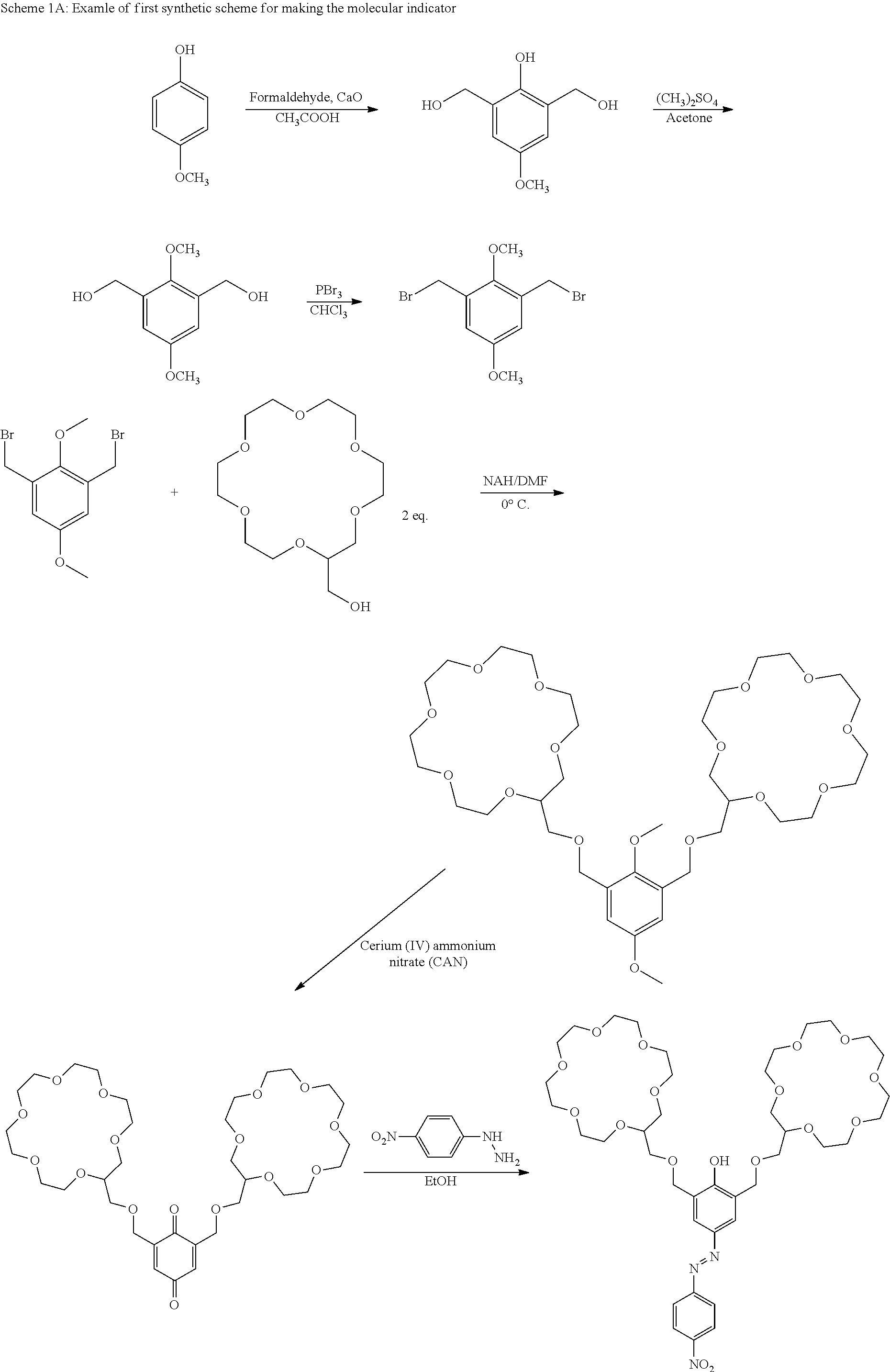

A second process for synthesizing the molecular indicator is shown by the following steps in Scheme 2.

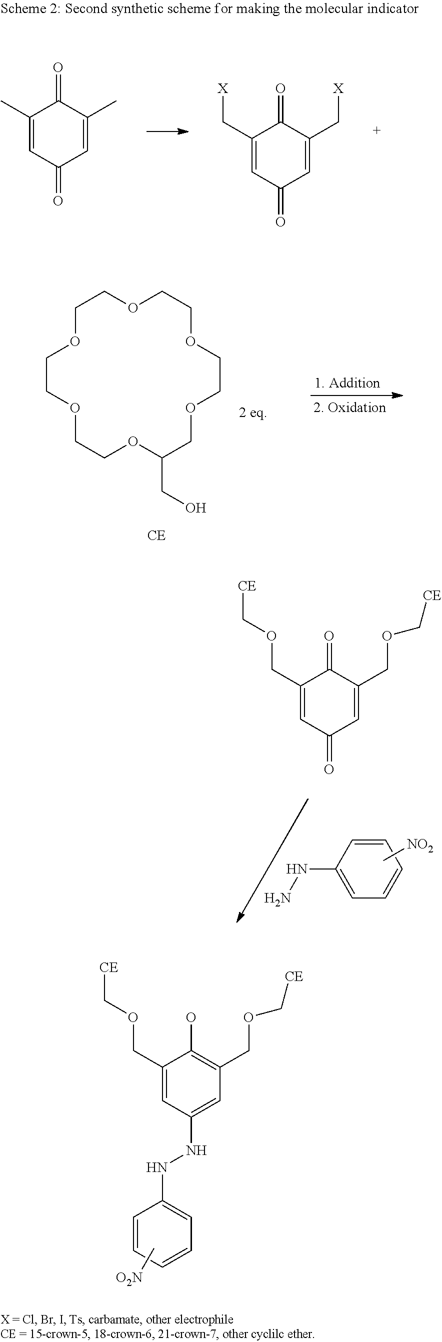

The following Scheme 2A shows an example of the second synthetic scheme for making the molecular indicator. This scheme involves brominating the 3, 6 dimethyl quinone with NBS (N-bromosuccinimide), followed by addition of the crown ether moiety. Subsequent incorporation of the azo-dye functional group follows the synthesis of the his crown quinone compound.

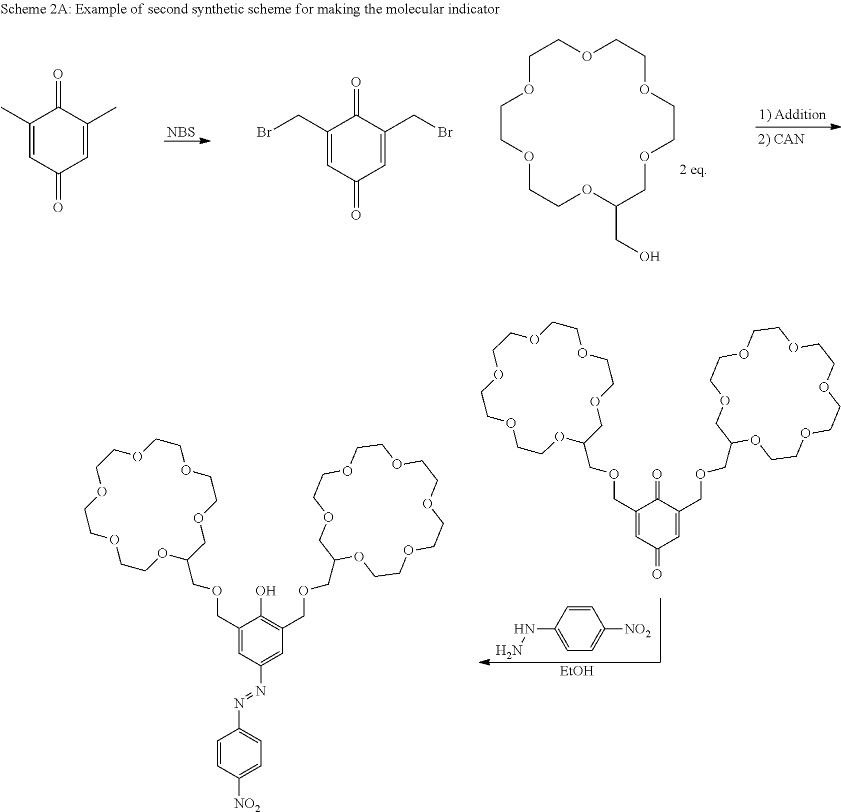

A third process for synthesizing the molecular indicator is shown by the following steps in Scheme 3.

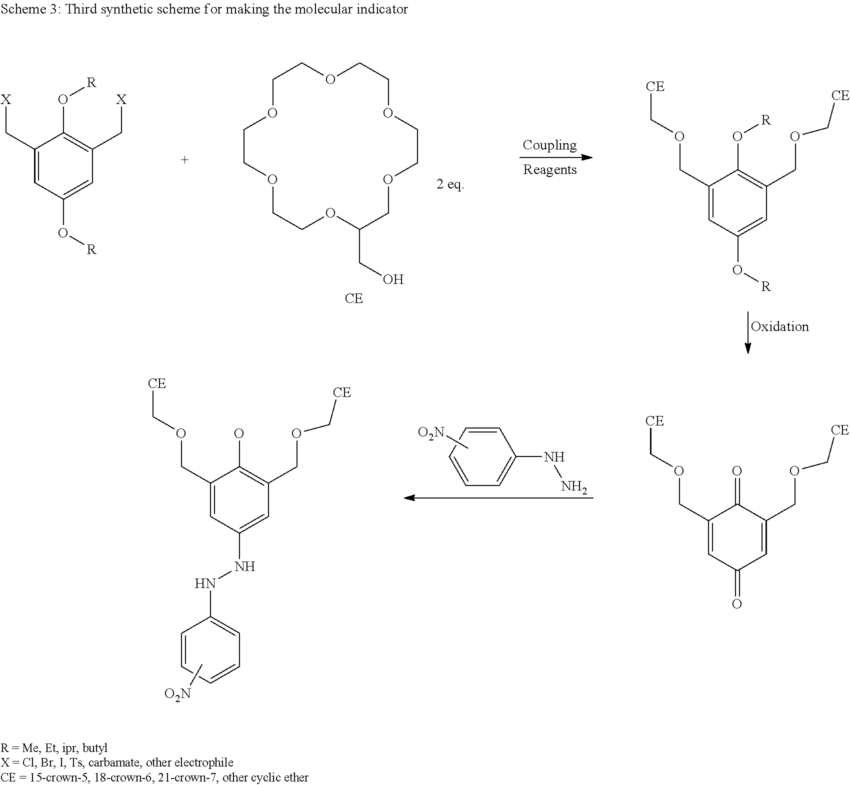

The following Scheme 3A shows an example of the third synthetic scheme for making the molecular indicator. This scheme involves the use of the Mitsunobu reaction to couple the alcohol functionality.

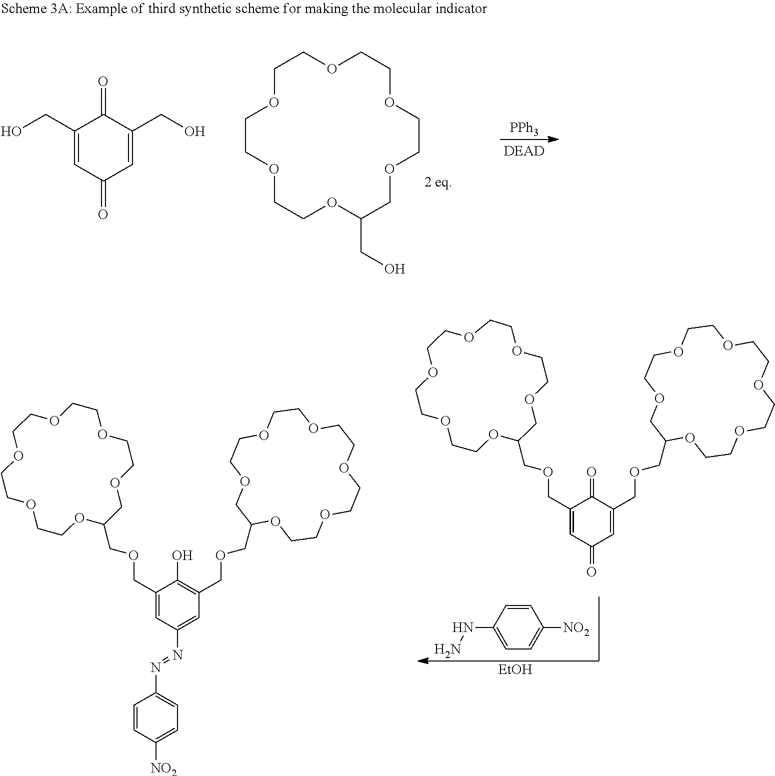

A fourth process for synthesizing the molecular indicator is shown by the following steps in Scheme 4.

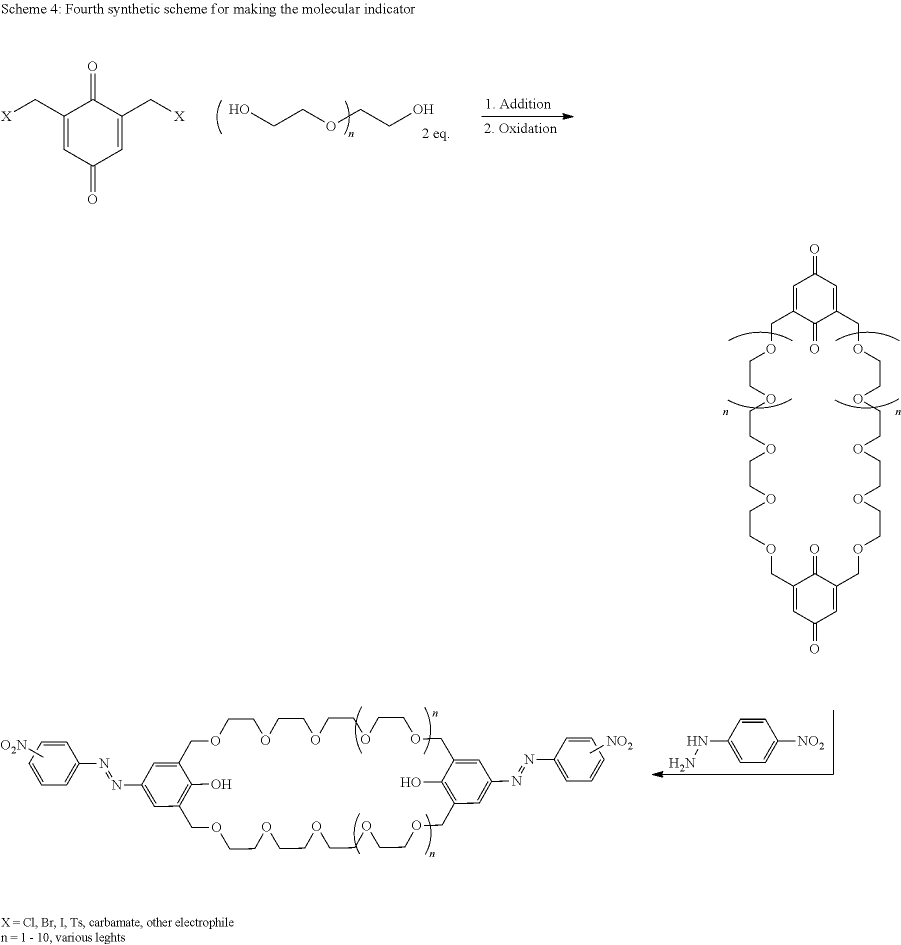

The following Scheme 4A shows an example of the fourth synthetic scheme for making the molecular indicator. This scheme creates crown ether-like functionality and involves addition of 2 equivalents of [n]ethylene glycol to 1 equivalent of the brominated quinone. This is followed by the addition of another equivalent of the brominated quinone to close the ring. Subsequent modification of the bis-quinone compound with 2 equivalents of the nitro azobenzene gives a di-substituted indicator compound.

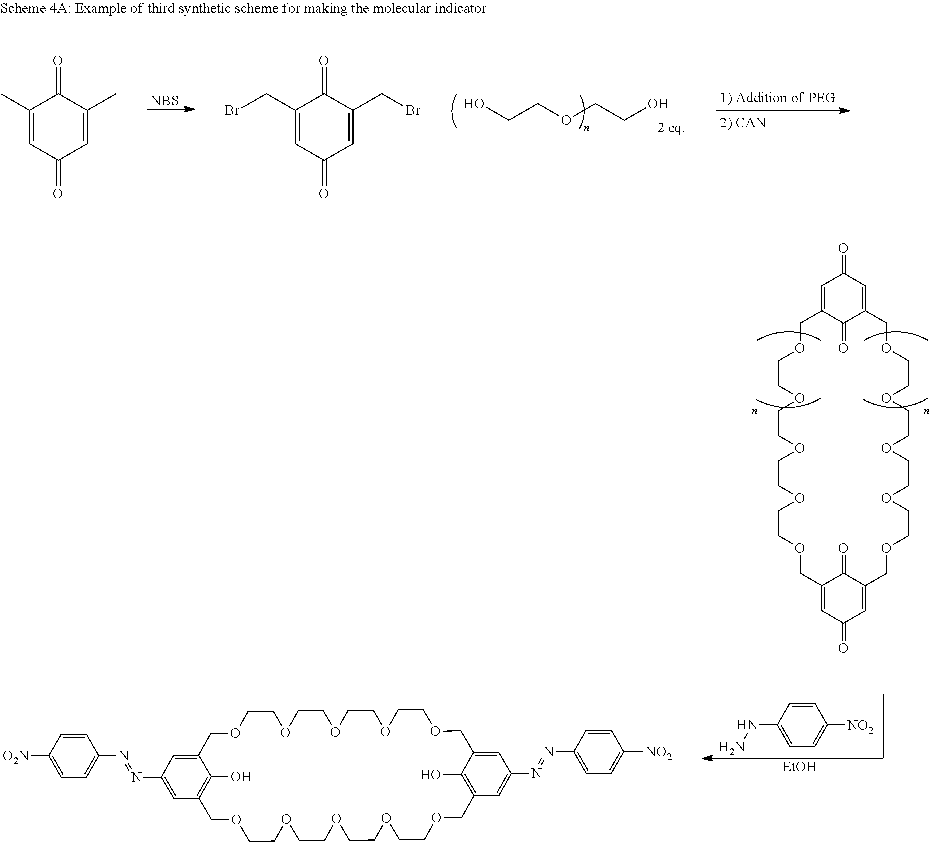

In accordance with the provisions of the patent statutes, the principle and mode of operation of the molecular indicator have been described in relation to preferred embodiments. However, it must be understood that it may be practiced otherwise than as specifically described without departing from its spirit or scope.

What is claimed is:

1. A molecular indicator for detecting primary diamines comprising an indicator structure coupled to (a) a bis-crown ether structure or (b) a structure having a bis-crown ether-like functionality.

2. The molecular indicator of claim 1, wherein the indicator structure comprises a colorimetric indicator.

3. The molecular indicator of claim 1, wherein the indicator structure is coupled to a bis-crown ether structure.

4. The molecular indicator of claim 1, wherein primary diamines are biogenic amines produced by meat spoilage.

5. The molecular indicator of claim 1, wherein the molecular indicator can be included with a substrate.

6. The molecular indicator of claim 5, wherein the molecular indicator can be included inside the substrate or on a surface of the substrate.

7. The molecular indicator of claim 5, wherein the substrate comprises paper, paperboard, polymer, plastic, cotton, resin, glass, fiberglass, or textile fabric.

8. The molecular indicator of claim 5, wherein the molecular indicator can be included inside a substrate in the form of a polymer matrix.

9. A package for detecting an analyte emitted by a packaged object comprising:
a packaging material for the object; and
a molecular indicator included with the packaging material for detecting the analyte, the molecular indicator comprising an indicator structure coupled to a trapping structure for trapping the analyte, the molecular indicator being highly specific for the analyte, and the trapping of the analyte by the molecular indicator avoiding reactive chemistry by-products.

10. The package of claim 9, wherein the packaged object is packaged food and the molecular indicator detects food spoilage.

11. The package of claim 10, wherein the molecular indicator detects primary diamines from food spoilage, and the molecular indicator comprises an indicator structure coupled to (a) a bis-crown ether structure or (b) a structure having a bis-crown ether-like functionality.

12. The package of claim 11, wherein the indicator structure is coupled to a bis-crown ether structure.

13. The package of claim 11, wherein the indicator structure comprises a colorimetric indicator.

14. The package of claim 9, wherein the analyte is a first analyte and the molecular indicator is a first molecular indicator, and further comprising a second molecular indicator as described in claim 9 for detecting a second analyte.

15. The package of claim 9, wherein the molecular indicator is a first molecular indicator, and further comprising a second molecular indicator as described in claim 9, the second molecular indicator cooperating with the first molecular indicator to enhance detection of the analyte.

16. The package of claim 15, wherein the first and second molecular indicators are different colorimetric indicators that cooperate to enhance visual detection of the analyte.

17. The package of claim 9 wherein the packaging material further includes an additive for enhancing the detection of the analyte.

18. A process of synthesizing a molecular indicator comprising the following scheme:

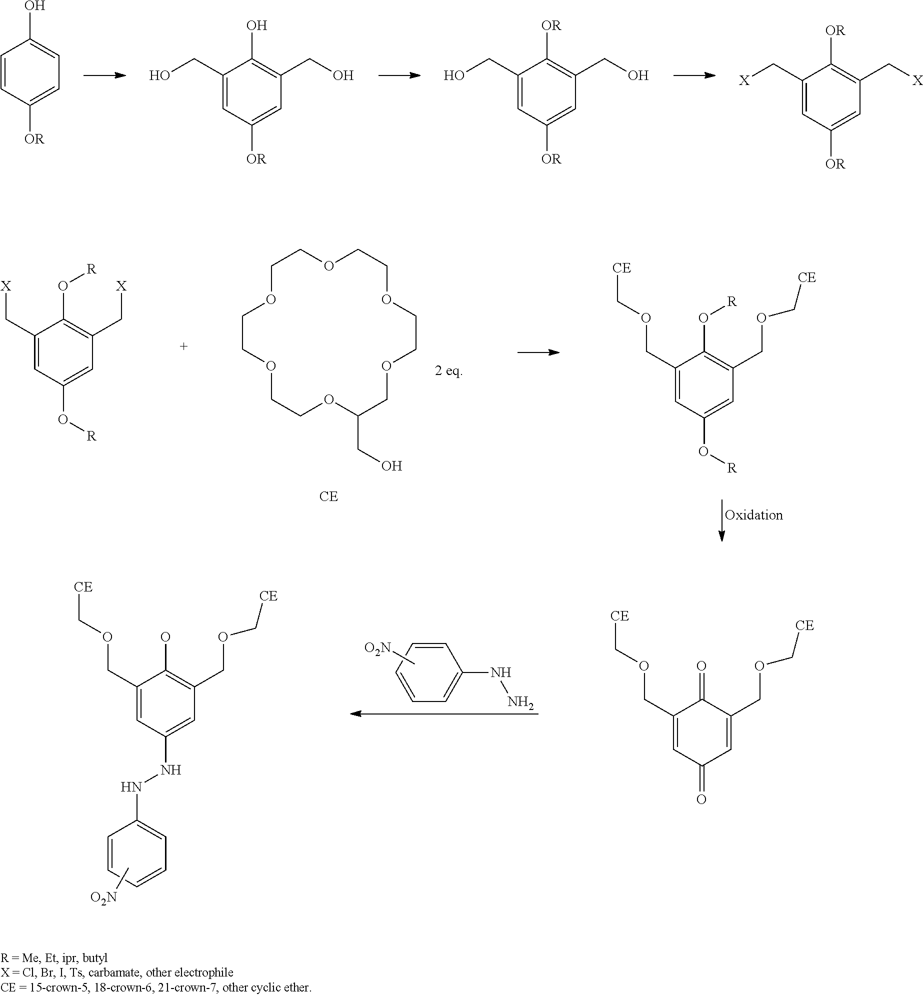

19. A process of synthesizing a molecular indicator comprising the following scheme:

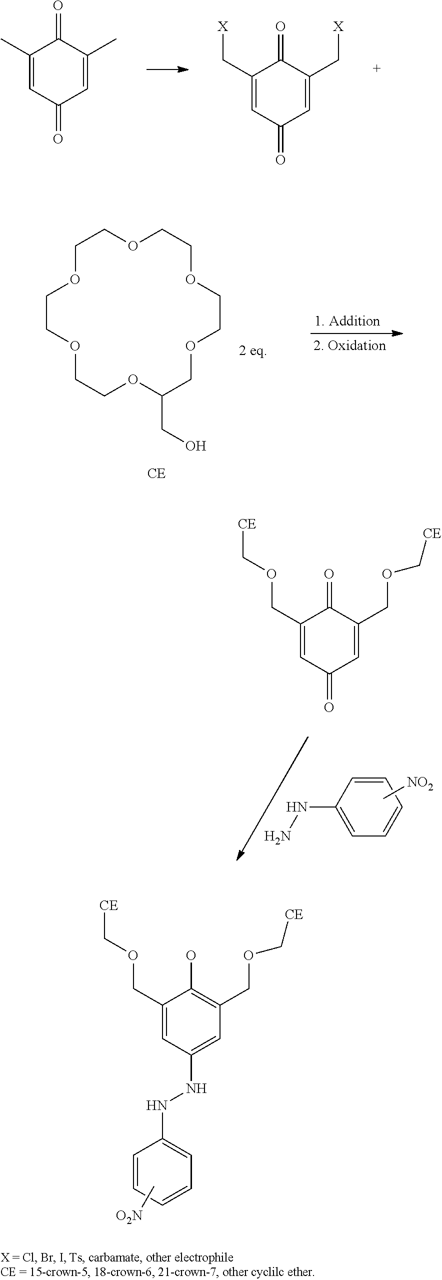

20. A process of synthesizing a molecular indicator comprising the following scheme:

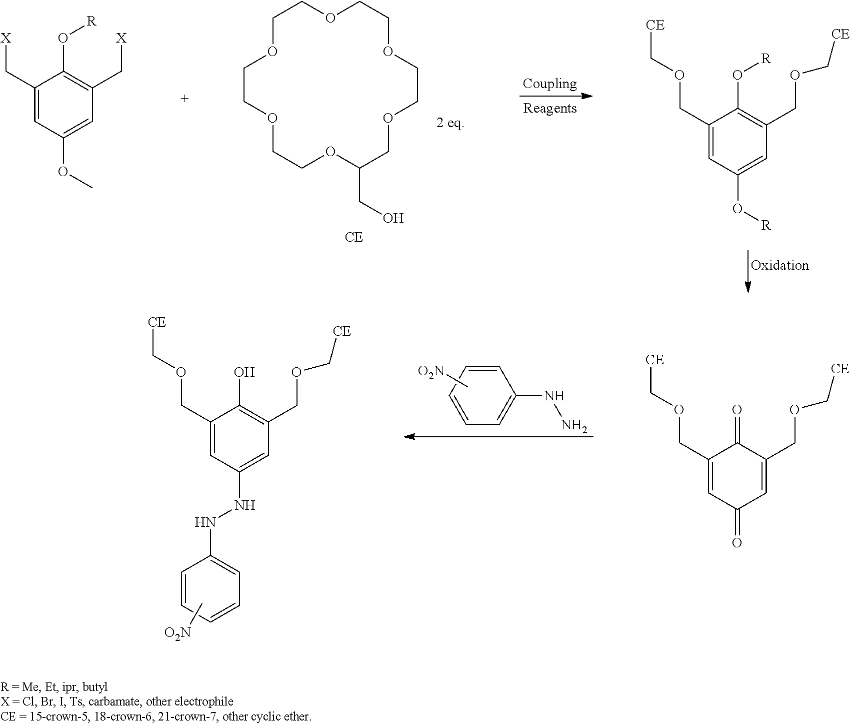

21. A process of synthesizing a molecular indicator comprising the following scheme: [chemical structure]
